(19) United States
(12) Patent Application Publication
(10) Pub. No.: US 2025/0235094 A1
(43) Pub. Date: Jul. 24, 2025

(54) MODEL EYE WITH PATHOLOGY

(71) Applicant: Verily Life Sciences LLC, Dallas, TX (US)

(72) Inventors: Bo Lu, Santa Clara, CA (US); Sam Kavusi, Menlo Park, CA (US); Dar Bahatt, Foster City, CA (US)

(21) Appl. No.: 18/794,984

(22) Filed: Aug. 5, 2024

Related U.S. Application Data
(60) Provisional application No. 63/623,569, filed on Jan. 22, 2024.

(51) Int. Cl. A61B 3/00; G06T 7/00

(52) U.S. Cl. CPC A61B 3/0025; G06T 7/00; G06T 2207/30041

(57) ABSTRACT

A model eye for evaluating a retinal imaging system includes a retinal cup, a corneal plate, and a dummy retina. The retinal cup includes an interior concave surface. The corneal plate mates to the retinal cup to define an interior cavity with the interior concave surface of the retinal cup. The dummy retina is formed from a flexible material layer attached to the interior concave surface. The dummy retina mimics a human retina and the flexible material layer includes a plurality of peripheral notches in a perimeter of the flexible material layer to aid the flexible material layer to conform to the interior concave surface.

### CROSS-REFERENCE TO RELATED APPLICATIONS

This application claims the benefit of U.S. Application No. 63/623,569, filed on Jan. 22, 2024, the contents of which are incorporated herein by reference.

### TECHNICAL FIELD

This disclosure relates generally to model eyes, and in particular but not exclusively, relates to model eyes that mimic pathologies for evaluating retinal camera systems.

### BACKGROUND INFORMATION

A model eye can be a valuable standalone product for the characterization, evaluation, and testing of fundus cameras. Use cases for a model eye include obtaining actionable feedback during the design of a fundus camera, evaluating the capabilities and function of a fundus camera, benchmarking fundus camera, validating the correct manufacture and assembly of a fundus camera, training the end user of the fundus camera, troubleshooting a fundus camera, etc.

Not all human eyes are disease free and a core mission for using retinal cameras is to monitor eyes for early onset of diseases or detect and diagnose advanced stage pathologies present in a diseased eye. To this end, a model eye that is capable of imitating a diverse spectrum of human eyes, including their diseases, will further the development, evaluation, and use of fundus/retinal cameras systems.

### BRIEF DESCRIPTION OF THE DRAWINGS

Non-limiting and non-exhaustive embodiments of the invention are described with reference to the following figures, wherein like reference numerals refer to like parts throughout the various views unless otherwise specified. Not all instances of an element are necessarily labeled so as not to clutter the drawings where appropriate. The drawings are not necessarily to scale, emphasis instead being placed upon illustrating the principles being described.

FIG. 1 is an exploded perspective view illustration of a model eye capable of mimicking pathologies, in accordance with an embodiment of the disclosure.

FIG. 2A is an exploded perspective view illustration of a dummy retina capable of mimicking pathologies, in accordance with an embodiment of the disclosure.

FIG. 2B is a perspective view illustration of a dummy retina capable of mimicking pathologies, in accordance with an embodiment of the disclosure.

FIGS. 2C and 2D are perspective view illustrations depicting placement of the dummy retina into a retinal cup of a model eye, in accordance with an embodiment of the disclosure.

FIG. 3A is a perspective view illustration of a dummy retina mimicking a pathology, in accordance with an embodiment of the disclosure.

FIG. 3B illustrates a screen-printing technique for forming pathologies on the dummy retina of a model eye, in accordance with an embodiment of the disclosure.

FIG. 3C illustrates a transfer-printing technique for forming pathologies on the dummy retina of a model eye, in accordance with an embodiment of the disclosure.

FIG. 4 illustrates a technique for fabricating a dummy crystalline lens with a nuclear cataract, in accordance with an embodiment of the disclosure.

FIG. 5 illustrates a technique for fabricating a dummy crystalline lens with a subcapsular cataract, in accordance with an embodiment of the disclosure.

FIG. 6 illustrates a technique for generating a photorealistic model eye, in accordance with an embodiment of the disclosure.

FIG. 7 illustrates a technique for generating a photorealistic model eye mimicking different retinal pathologies, in accordance with an embodiment of the disclosure.

### DETAILED DESCRIPTION

Embodiments of a system, apparatus, and method of manufacture of a model eye capable of mimicking various ophthalmic pathologies are described herein. In the following description numerous specific details are set forth to provide a thorough understanding of the embodiments. One skilled in the relevant art will recognize, however, that the techniques described herein can be practiced without one or more of the specific details, or with other methods, components, materials, etc. In other instances, well-known structures, materials, or operations are not shown or described in detail to avoid obscuring certain aspects.

Reference throughout this specification to “one embodiment” or “an embodiment” means that a particular feature, structure, or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

Embodiments of the model eye described herein include features and subcomponents adapted to mimic various ophthalmic pathologies. For example, various embodiments are capable of mimicking one or more pathologies including exudates and microaneurysm for diabetic retinopathy, drusen for age-related macular degeneration, hemorrhages, or cloudy crystalline lenses for nuclear cataracts or posterior/anterior subcapsular cataracts. By mimicking these various pathologies on a dummy retina or dummy crystalline lens, the model eye can be used to evaluate, validate, or benchmark the capabilities of various fundus cameras or other retinal imaging systems. Additionally, the model eye can be used to train technicians, clinicians, or other healthcare workers to operate retinal imaging system equipment and identify ophthalmic pathologies.

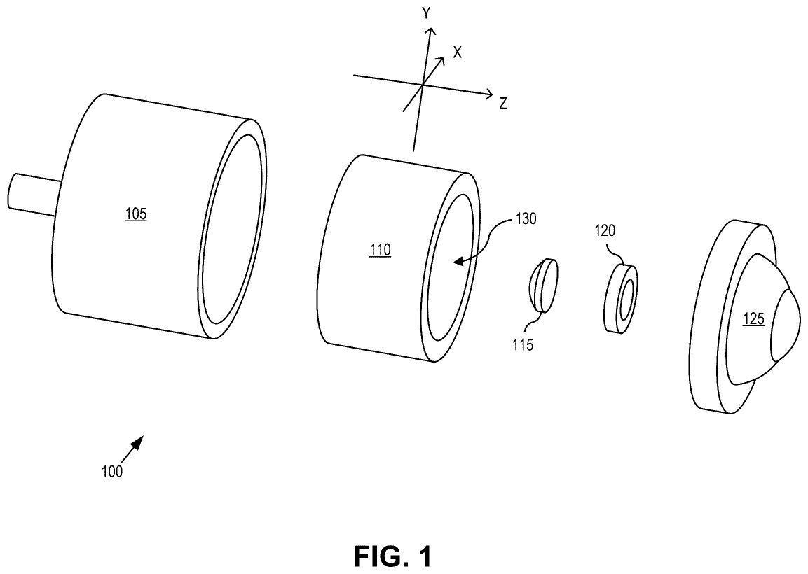

FIG. 1 is an exploded perspective view illustration of a model eye 100 capable of mimicking various ophthalmic pathologies, in accordance with an embodiment of the disclosure. The illustrated embodiment of model eye 100 includes an outer holder 105, a retinal cup 110, a dummy crystalline lens 115, an iris insert 120, and a corneal plate 125. When assembled, retinal cup 110 slides into outer holder 105 while corneal plate 125 clamps over the top of retinal cup 110 and is held in place by outer holder 105. Dummy crystalline lens 115 attaches to iris insert 120 while the combined iris insert 120 and dummy crystalline lens 115 attach to the inside of corneal plate 125. Retinal cup 110 at least partially defines an interior cavity 130, which includes an interior concave surface where a dummy retina is attached to mimic the retina of a human eye. When corneal plate 125 is clamped to retinal cup 110, interior cavity 130 is sealed and defined by retinal cup 110 and corneal plate 125. The interior cavity 130 may be sealed with an o-ring positioned between retinal cup 110 and corneal plate 125 and operate as a fluid chamber when filled with a liquid (e.g., distilled water, saline solution, etc.) to represent the vitreous humor of the human eye. In the various embodiments described below, a dummy retina is conformally attached to the interior concave surface of retinal cup 110. The dummy retina may mimic various retinal pathologies. Similarly, in various embodiments, dummy crystalline lens 115 may be clouded to mimic various types of cataracts.

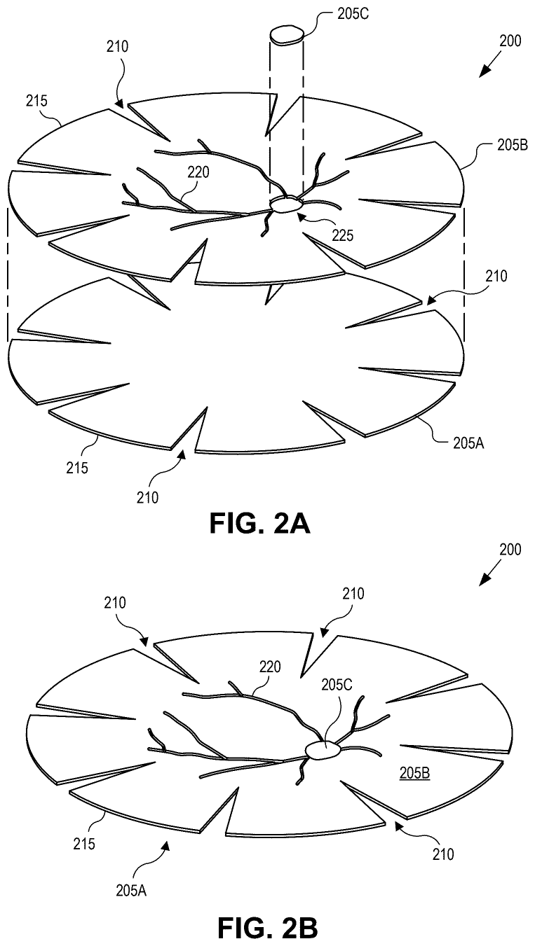

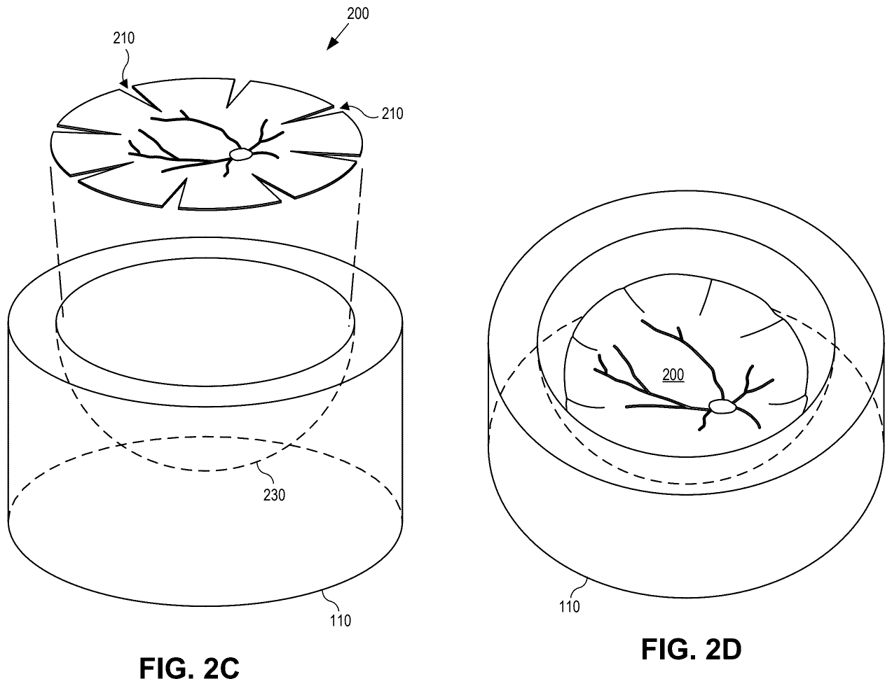

FIGS. 2A and 2B illustrated a dummy retina 200 capable of mimicking retinal pathologies, in accordance with an embodiment of the disclosure. FIG. 2A is an exploded perspective view of dummy retinal 200 illustrating the multiple flexible material layers 205A, B, and C (collectively referred to as flexible material layers 205) while FIG. 2B illustrates flexible material layers 205 collapsed together into a laminated stack.

As mentioned above, interior cavity 130 may be filled with a liquid (e.g., water, saline, etc.) to mimic the vitreous humor of a human eye. In the illustrated embodiment, dummy retina 200 is adhered to an interior concave surface of retinal cup 110 positioned in direct contact with the fill liquid. Accordingly, flexible material layers 205 may be fabricated from a laminated stack of plastic layers (e.g., adhesive colored vinyl sheets) that is resistant to delaminating in the presence of water. Flexible material layer 205A is a base layer adhered to the interior concave surface and has a first color (e.g., dark red) that mimics the color of blood vessels in a human retina. Flexible material layer 205B is a top layer disposed over the base layer and includes cutouts 220 mimicking a retinal vasculature shape (e.g., dendritic shape). The top layer is an opaque layer having a second color, different than the first color of the base layer. The second color of the top layer is selected to mimic a background color of a human retina. In one embodiment, the second color of the top layer is a lighter red color than the darker red color of the base layer. Cutouts 220 permit the darker red color to show through the opaque top layer such that cutouts 220 resemble a human retinal vasculature in shape and color. Flexible material layer 205C is a third layer having a shape and a color (e.g., yellowish) to mimic an optic disc of a human retina. Flexible material layer 205C may also be opaque like the base and top layers. In the illustrated embodiment, flexible material layer 205C is adhered into a corresponding optic disc cutout 225 in the top layer (flexible material layer 205B). In other embodiments, flexible material layer 205C may be adhered onto the top layer that omits optic disc cutout 225.

In the illustrated embodiment, flexible material layers 205A and 205B each include a plurality of peripheral notches 210 in perimeters 215 of flexible material layers 205A and 205B. Peripheral notches 210 are sacrificial “stitching cuts” that aid with three-dimensional (3D) forming of the laminated stack to the interior concave surface of retinal cup 110. FIGS. 2C and 2D illustrate this 3D forming and conformal placement of dummy retina 200 onto interior concave surface 230 of retinal cup 110, in accordance with an embodiment of the disclosure. In one embodiment, flexible material layers 205 are color vinyl adhesive layers that are laser cut to form cutouts 210, optic disc cutout 225, and the overall shape of perimeters 215.

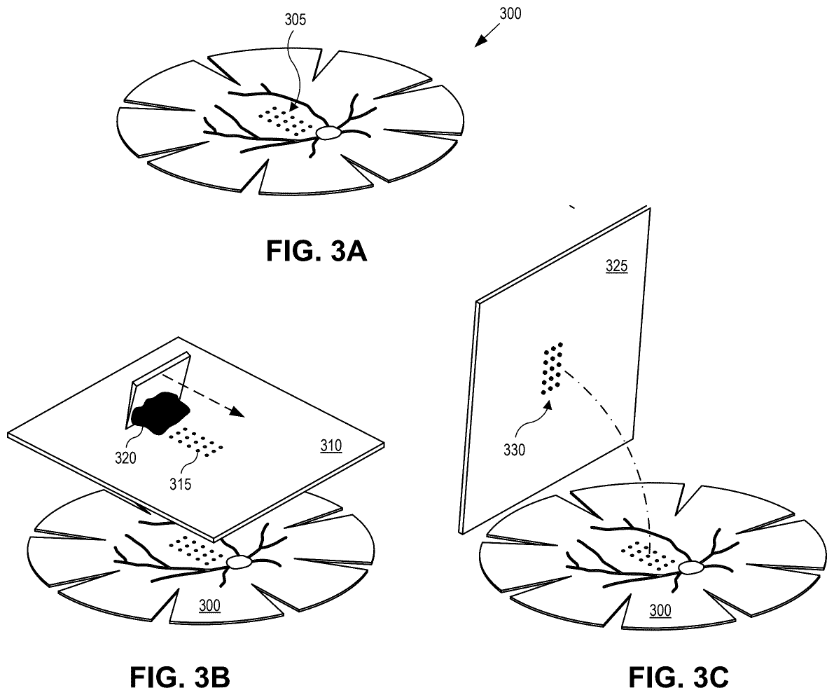

As mentioned, dummy retina 200 is suitable for mimicking retinal pathologies. FIG. 3A is a perspective view illustration of dummy retina 300 that includes one or more features 305 mimicking one or more retinal pathologies, in accordance with an embodiment of the disclosure. Dummy retina 300 is similar to dummy retina 200 except for the addition of features 305 disposed thereon. Features 305 may be formed using a variety of different techniques. In some embodiments, features 305 may be printed onto the top layer of dummy retina 300 using an oil-based paint. The oil-based paint is resistant to dissolving in the presence of a water-based fill fluid in interior cavity 130. In other embodiments, features 305 may be fabricated using a low power UV laser for direct laser scribing. The size, shape, and color of features 305 may be selected to resemble various retinal pathologies such as exudates, drusens, microaneurysms, or hemorrhages. For example, exudates may be yellow, drusens may be white, hemorrhages may be brown, and microaneurysms may be a darker tint of the background retinal color.

FIG. 3B illustrates a screen-printing technique for forming retinal pathologies on dummy retina 300, in accordance with an embodiment of the disclosure. As illustrated, a thin film sacrificial screen 310 is laser cut to form small openings 315. Screen 310 may be fabricated using Kapton tape. Screen 310 is adhered over the top layer of dummy retina 300 and a liquid paint 320 (e.g., oil-based paint) is squeegeed over openings 315. After curing/drying, the screen 310 is removed leaving behind a pathology pattern printed over the top of dummy retinal 300. With screen printing, features 305 may be printed to mimic pathologies as small as 75 um.

FIG. 3C illustrates a transfer-printing technique for forming retinal pathologies on dummy retina 300, in accordance with another embodiment of the disclosure. As illustrated, a stamp 325 is used to transfer a paint pattern onto the top layer of dummy retina 300. Stamp 325 may be fabricated from silicon (e.g., polydimethylsiloxane or PDMS). Stamp 325 includes miniaturized posts 330 that assume the size, shape, and pattern of a retinal pathology. Stamp 325 is pressed into a liquid paint (e.g., oil-based paint) and then pressed onto the top of dummy retina 300. After curing/drying, the pathology pattern remains on the top layer of dummy retinal 300. With transfer-printing, features 305 may be printed to mimic pathologies as small as 100 um.

In yet another embodiment, features 305 may be direct laser scribed onto the top of dummy retina 300. In direct laser scribing, a low power UV laser is used to char the surface of dummy retina 300 with the pathology pattern. Direct laser scribing enables forming pathologies as small as 15 um. Direct laser scribing is well suited to form features 305 that mimic microaneurysms having low contrast ranging in sizes from 15 um to 60 um.

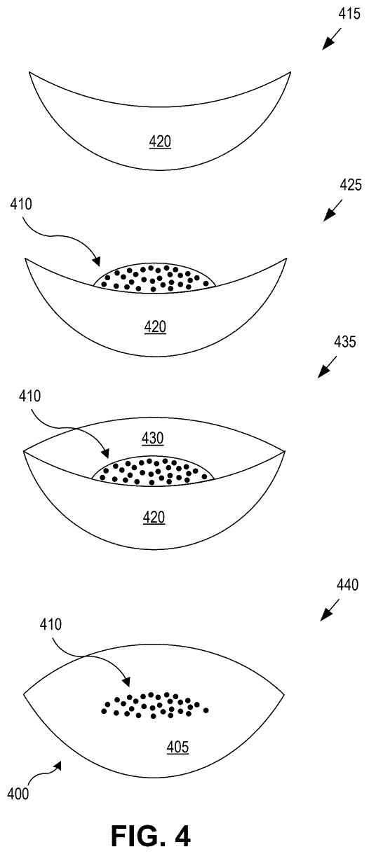

In addition to mimicking retinal pathologies, embodiments of model eye 100 may also mimic other ophthalmic pathologies such as cataracts. FIG. 4 illustrates a technique for fabricating a dummy crystalline lens 400 with a nuclear cataract, in accordance with an embodiment of the disclosure. Dummy crystalline lens 400 is one possible implementation of dummy crystalline lens 115 illustrated in FIG. 1. Dummy crystalline lens 400 includes a clear material 405 having a frosted glass suspension 410 disposed as an interior deposit within clear material 405, which mimics a nuclear cataract.

As illustrated in FIG. 4, dummy crystalline lens 400 may be fabricated in multiple stages. In a first stage (illustration 415), a bottom portion 420 is molded and cured from a clear material. Bottom portion 420 is formed into a crescent-like shape. The clear material may include PDMS, epoxy, or other clear materials that are compatible with the solvent in frosted glass suspension 410. Once bottom portion 420 is formed and cured, a drop of frosted glass suspension 410 is deposited onto the middle of the concave side of bottom portion 420 (illustration 425). In one embodiment, the frosted glass suspension 410 is a mixture of the clear material precursor (e.g., PDMS liquid precursor) mixed with a semi-transparent frosted glass material (e.g., Krylon's semi-transparent Frosted Glass spray). The frosted glass suspension 410 is then cured hard. Next, a top portion 430 is molded over bottom portion 420 and the cured frosted glass suspension 410 (illustration 435). Top portion 430 is formed from the same clear material as bottom portion 420. Top portion 430 is then also cured forming the completed dummy crystalline lens 400 (illustration 440) having a center cloudy region formed by frosted glass suspension 410 surrounded by clear material 405. The center cloudy region mimics a nuclear cataract. The different stages of cataract progression may be simulated by varying the size of frosted glass suspension 410 and/or the transparency of frosted glass suspension 410 by increasing the ratio of frosted glass to the clear material precursor.

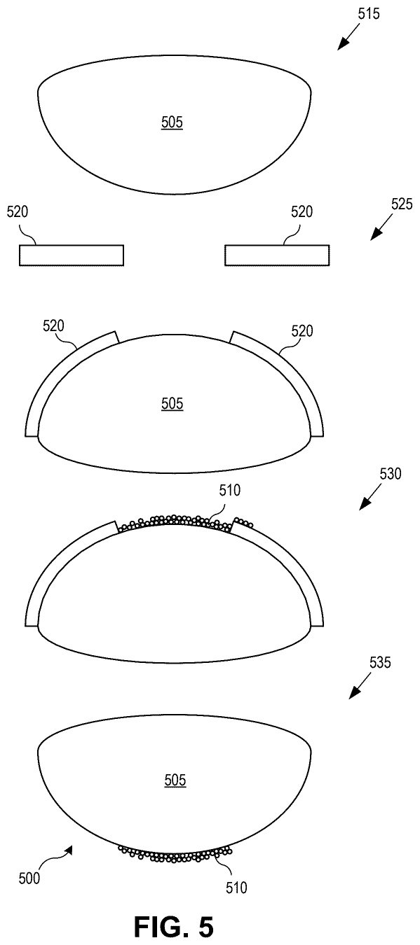

While FIG. 4 illustrates a dummy crystalline lens with a nuclear cataract pathology, FIG. 5 illustrates a technique for fabricating a dummy crystalline lens 500 with a subcapsular cataract, in accordance with an embodiment of the disclosure. Dummy crystalline lens 500 is one possible implementation of dummy crystalline lens 115 illustrated in FIG. 1. Dummy crystalline lens 500 includes a clear material 505 having a frosted glass suspension 510 disposed as a coating on an outer surface of clear material 505, which mimics a subcapsular cataract (e.g., anterior subcapsular cataract, posterior subscapular cataract, or both).

As illustrated in FIG. 5, dummy crystalline lens 500 may be fabricated in multiple stages. In a first stage (illustration 515), the lens body shape is molded and cured from clear material 505. Clear material 505 may include polymethyl methacrylate (PMMA), PDMS, epoxy, glass, or other clear materials. A mask 520 is formed and adhered to cured clear material 505 (illustration 525). In one embodiment, mask 520 is Kapton tape laser cut to form an aperture. After mask 520 is formed on clear material 505, the frosted glass suspension 510 is coated (e.g., sprayed or brushed) over the aperture in mask 520 (illustration 530). The frost glass suspension 510 may be a semi-transparent frosted glass material, such as Krylon's semi-transparent Frosted Glass spray. The frosted glass suspension 510 is then cured and mask 520 removed to form dummy crystalline lens 500 (illustration 535). The frosted portion of dummy crystalline lens 500 mimics a subcapsular cataract. The frosted portion may be formed on the anterior and/or posterior sides of clear material 505 to mimic anterior and/or posterior subcapsular cataracts on dummy crystalline lens 500. Of course, the technique illustrated in FIG. 5 may be applied to the dummy crystalline lens 400 to mimic a crystalline lens that includes both subcapsular and nuclear cataracts. The thickness of frost glass suspension 510 and/or the size of the aperture may be varied to represent different stages of cataract progression.

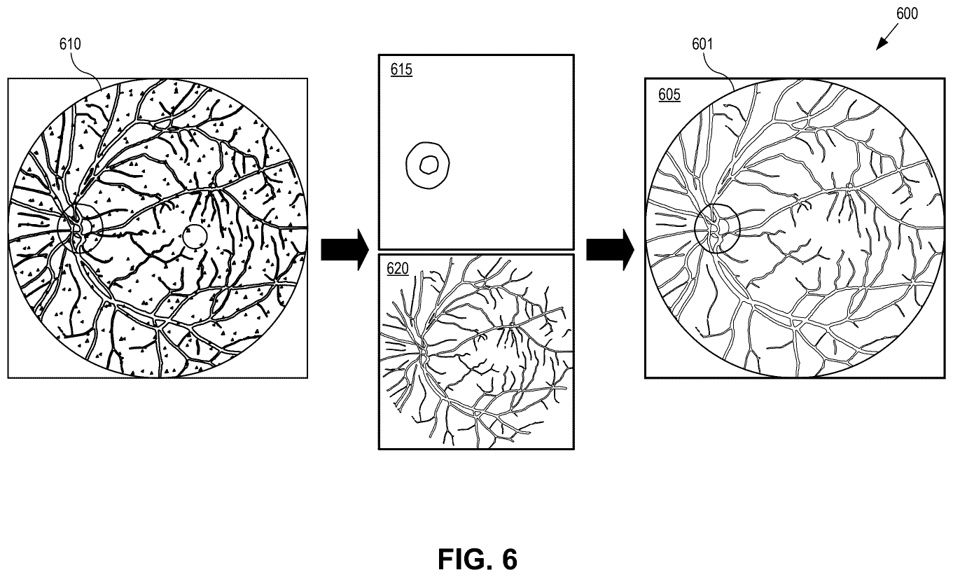

FIG. 6 illustrates a technique for generating a model eye having a photorealistic dummy retina 600, in accordance with an embodiment of the disclosure. Photorealistic dummy retina 600 is achieved by printing a simplified retinal image 601 on a flexible material layer 605. Flexible material layer 605 may then be trimmed to shape and adhered to the interior concave surface 230 of retinal cup 110 as a sort of plastic sticker. In one embodiment, flexible material layer 605 is a vinyl sheet (e.g., white vinyl sheet) and may also be trimmed to include peripheral notches 210, as illustrated in FIG. 2A, to facilitate conformal adherence to interior concave surface 230. Simplified retinal image 601 may be printed (e.g., laser printer) at a 720 dpi resolution onto flexible material layer 605, though the retinal image source file may be a higher resolution itself. Of course, higher resolution printers may be used.

Simplified retinal image 601 is generated by using semantic segmentation on a real retinal image 610 to extract anatomical features including the optic disc 615 and retinal vasculature 620. The extracted features are isolated and cleaned/simplified to eliminate noise present in the real retinal image 610. A deep learning convolutional neural network (CNN) may be used to identify and extract the anatomical features. These anatomical features are then recombined in the simplified retinal image 601. The simplified retinal image 601 eliminates the graininess and artifacts present in the real retinal image 610.

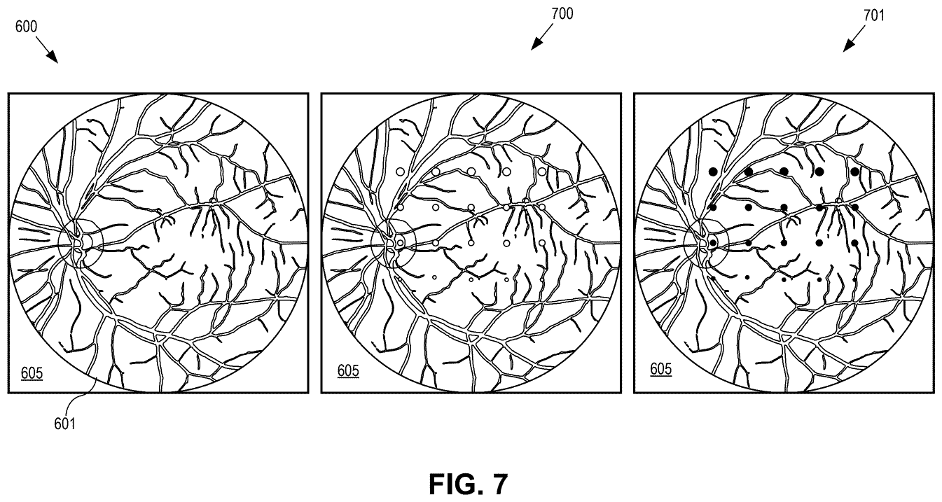

FIG. 7 illustrates how photorealistic dummy retina 600 may be altered by adding one or more retinal pathologies to generate photorealistic dummy retinas 700 or 701 that mimic different pathologies, in accordance with embodiments of the disclosure. The pathologies in photorealistic dummy retinas 700 or 701 may include exudates, drusens, microaneurysms, hemorrhages, or otherwise. The pathologies may be incorporated into the simplified retinal image or applied onto the dummy retina using any of the techniques described above (e.g., screen-printing, transfer-printing, direct laser scribing, or combinations thereof).

The above description of illustrated embodiments of the invention, including what is described in the Abstract, is not intended to be exhaustive or to limit the invention to the precise forms disclosed. While specific embodiments of, and examples for, the invention are described herein for illustrative purposes, various modifications are possible within the scope of the invention, as those skilled in the relevant art will recognize.

These modifications can be made to the invention in light of the above detailed description. The terms used in the following claims should not be construed to limit the invention to the specific embodiments disclosed in the specification. Rather, the scope of the invention is to be determined entirely by the following claims, which are to be construed in accordance with established doctrines of claim interpretation.

What is claimed is:

1. A model eye for evaluating a retinal imaging system, the model eye comprising:
a retinal cup including an interior concave surface;
a corneal plate that mates to the retinal cup to define an interior cavity with the interior concave surface of the retinal cup; and
a dummy retina formed from a flexible material layer attached to the interior concave surface, wherein the dummy retina mimics a human retina and wherein the flexible material layer includes a plurality of peripheral notches in a perimeter of the flexible material layer to aid the flexible material layer to conform to the interior concave surface.

2. The model eye of claim 1, wherein the flexible material layer comprises a plastic layer.

3. The model eye of claim 1, wherein the dummy retina comprises a laminated stack of multiple flexible material layers, including the flexible material layer, adhered to the interior concave surface.

4. The model eye of claim 3, wherein the laminated stack comprises:
a base layer adhered to the interior concave surface having a first color to mimic blood vessels in the human retina; and
a top layer disposed over the base layer and including cutouts mimicking a retinal vasculature shape so that the first color of the base layer shows through the top layer in the cutouts.

5. The model eye of claim 4, wherein the top layer comprises an opaque layer having a second color, different than the first color, that mimics a background color of the human retina.

6. The model eye of claim 5, wherein the top layer comprises a lighter red color than a darker red color of the base layer, wherein the top layer and the base layer both comprise adhesive vinyl sheets.

7. The model eye of claim 4, wherein the laminated stack further comprises:
a third layer having a shape and a color to mimic an optic disc of the human retina.

8. The model eye of claim 1, wherein the dummy retina includes one or more features formed on the flexible material layer of the dummy retina to mimic a retinal pathology.

9. The model eye of claim 8, wherein the one or more features are printed using an oil-based paint and have a size, a shape, and a color to mimic one or more of a microaneurysm, an exudate, or a drusen.

10. The model eye of claim 8, wherein the one or more features comprise a microaneurysm that is direct laser scribed onto the dummy retina.

11. The model eye of claim 1, further comprising:
a dummy crystalline lens that mounts within the interior cavity, wherein the dummy crystalline lens includes a clear material having a frosted glass suspension disposed in or on the clear material of the dummy crystalline lens to mimic a cataract pathology.

12. The model eye of claim 11, wherein the frosted glass suspension is disposed as a coating on an outer surface of the clear material to mimic at least one of an anterior subcapsular cataract or a posterior subcapsular cataract.

13. The model eye of claim 11, wherein the frost glass suspension is disposed as an interior deposit within the clear material to mimic a nuclear cataract.

14. The model eye of claim 1, wherein the dummy retinal includes a retinal vasculature image and an optic disc image printed on the flexible material layer, wherein the retinal vasculature image and the optic disc image are generated from a semantic segmentation of a real image of the human retina.

15. A model eye comprising:
a retinal cup including an interior concave surface; and
a dummy retina formed from a laminated stack of flexible material layers adhered to the interior concave surface, wherein the laminated stack conforms to the interior concave surface and includes:
a base layer adhered to the interior concave surface having a first color to mimic blood vessels in a human retina; and
a top layer disposed over the base layer and including cutouts mimicking a retinal vasculature shape so that the first color of the base layer shows through the top layer in the cutouts.

16. The model eye of claim 15, wherein the flexible material layers each include a plurality of peripheral notches in a perimeter of each of the flexible material layers to aid the flexible material layers to conform to the interior concave surface.

17. The model eye of claim 15, wherein the top layer comprises an opaque layer having a second color that is lighter than the first color, which second color mimics a background color of the human retina.

18. The model eye of claim 15, wherein the laminated stack further comprises:
a third layer having a shape and a color to mimic an optic disc of the human retina.

19. The model eye of claim 15, wherein the dummy retina includes one or more features printed with an oil-based paint on the flexible material layer of the dummy retina to mimic one or more of a microaneurysm, an exudate, or a drusen of the human retina.

20. The model eye of claim 15, further comprising:
a dummy crystalline lens mounted within the model eye, wherein the dummy crystalline lens includes a clear material having a frosted glass suspension disposed in or on the clear material of the dummy crystalline lens to mimic a cataract pathology.
